(19) United States
(12) Patent Application Publication
(10) Pub. No.: US 2019/0304588 A1
(43) Pub. Date: Oct. 3, 2019

(54) FOOD INFORMATION PROCESSING METHOD AND APPARATUS

(71) Applicant: FOSHAN SHUNDE MIDEA ELECTRICAL HEATING APPLIANCES MANUFACTURING CO., LTD., Foshan (CN)

(72) Inventors: Pengbo REN, Foshan (CN); Baizhong MA, Foshan (CN)

(21) Appl. No.: 16/467,043

(22) PCT Filed: Nov. 16, 2017
(86) PCT No.: PCT/CN2017/111334

§ 371 Date: Jun. 6, 2019

(30) Foreign Application Priority Data
Dec. 21, 2016 (CN) 201611191208.1

(51) Int. Cl. G16H 20/60; G06Q 30/06

(52) U.S. Cl. CPC G16H 20/60; G06F 9/542; G06Q 30/0631

(57) ABSTRACT

Provided are a processing method and apparatus for food information. The method comprises: acquiring food information in a user's home; acquiring user information about the user, and generating recipe information matching the user information; providing the recipe information to the user; according to a confirmation instruction input by the user regarding the recipe information, determining whether the food information satisfies the food requirements of the recipe information; and if not, then ordering and purchasing the food needed for cooking according to the recipe information for the user. In the method, whether food information in a user's home satisfies food requirements of recipe information is judged, and the food needed for cooking according to the recipe information is purchased for the user, so that the trouble for the user to judge whether the food at home satisfies the food requirements of the recipe by himself/herself and the time spent purchasing the required food are reduced, thereby effectively improving the use experience of the user.

### FIELD

The disclosure relates to the field of food processing, and in particular to food information processing methods and apparatuses.

### BACKGROUND

With the continuous improvement of people's living standards, people's pursuit of delicious food is getting higher. Currently, users can search a variety of recipes from the Internet and cook food based on the recipes.

At present, after users obtain the recipes information that meeting their needs, the users need to confirm whether there is food needed for the recipes in the home, and need to go to the vegetable markets or the supermarkets for purchase, consequently, the users spend a long time in the cooking process, and inconvenience is brought to life.

### SUMMARY

The object of the invention is to solve at least one of the above technical problems to some extent.

To this endSome embodiments of the disclosure are to provide a food information processing method. According to the method, recipes and cooking information are supplied to dining users according to the number of diners and the dining area, thus, the dining users can cook food matching the number of diners according to the recipes and the cooking information, so that the individual needs of the dining users are met.

Some embodiments of the disclosure are to provide food information processing apparatus.

The food information processing method according to a first embodiment of the disclosure comprises the steps of obtaining food information in a user' home; obtaining user information about the user, and generating recipe information matching the user information; providing the recipe information for the user; determining whether the food information satisfies food material requirements of the recipe information according to a confirmation instruction input by the user regarding the recipe information; and if not, then ordering and purchasing the food needed for cooking according to the recipe information for the user.

According to the food information processing method provided by the embodiment of the disclosure, the recipe information is generated according to the user information of the user, whether food information in the home of the user meets the food requirements of the recipe information is determined when the user confirms the recipe information, and an order is placed for the user and the food required for cooking according to the recipe information is purchased when the food information in the home of the user does not meet the food requirements of the recipe information. Therefore, whether the food information in the home of the user meets the food requirements of the recipe information is determined, and the food needed for cooking according to the recipe information is purchased, so that the trouble for the user to determine whether the food in the home satisfies the food requirements of the recipe is reduced, and the time spent in purchasing the required food is shortened, and the user experience is effectively improved.

In an embodiment of the disclosure, the user information comprises at least one of dietary habit information, health information and weight information.

In an embodiment of the disclosure, the method further comprises the steps of obtaining dining demand information of the user; adjusting the recipe information according to the dining demand information, and thus determining whether the food information meets the information food requirements of the adjusted recipe information.

In an embodiment of the disclosure, the dining demand information comprises the number of diners and/or themes of dining demands, and the step of obtaining the dining demand information of the user comprises: receiving the number of diners and/or the themes of dining demands uploaded by the user through terminal equipment.

In one embodiment of the disclosure, before the step of ordering and purchasing the food needed for cooking according to the recipe information for the user, the method further comprises the steps of providing the user with link information for purchasing the food needed for cooking according to the recipe information; and receiving a confirmation instruction of the user for the link information, and executing the step of ordering and purchasing the food needed for cooking according to the recipe information for the user.

The food information processing device of a second embodiment of the disclosure comprises a first obtaining device used for obtaining food information in a user's home, a generating device used for obtaining user information of the user and generating recipe information matching the user information, a first providing device used for providing the recipe information for the user, a determining device used for determining whether the food information meets the food requirements of the recipe information according to a confirmation instruction input by the user regarding the recipe information, and a processing device used for ordering and purchasing the food needed for cooking according to the recipe information for the user when the food information is determined not to meet the food requirements of the recipe information.

According to the food information processing device provided by the embodiment of the disclosure, the recipe information is generated according to the user information of the user, whether food information in the home of the user meets the food requirements of the recipe information is judged when the user confirms the recipe information, and an order is placed for the user and the food needed for cooking according to the recipe information is purchased when the food information in the home of the user does not meet the food requirements of the recipe information. Therefore, whether the food information in the home of the user meets the food requirements of the recipe information is judged, and the food needed for cooking according to the recipe information is purchased, so that the trouble for the user to determine whether the food in the home satisfies the food requirements of a recipe is reduced, and the time spent in purchasing the required food is shortened, and the user experience is effectively improved.

In an embodiment of the disclosure, the user information comprises at least one of dietary habit information, health information and weight information.

In an embodiment of the disclosure, the apparatus further comprises:

A second obtaining device used for obtaining the dining demand information of the user; and

An adjustment device used for adjusting the recipe information according to the dining demand information so as to judge whether the food information meets the food requirements of the adjusted recipe information.

In an embodiment of the disclosure, the dining requirement information comprises the number of diners and/or themes of dining demands, and

The second obtaining device is used for receiving the number of diners and/or themes of dining demands uploaded by the user through terminal equipment.

In an embodiment of the disclosure, the apparatus further comprises:

A second providing device used for providing the user with link information for purchasing the food needed for cooking according to the recipe information;

In one embodiment, the processing device is specifically used for placing an order for a user according to a confirmation instruction of the user for the link information, and purchasing scant food materials needed for the cooking recipe information.

Additional aspects and advantages of the disclosure will be set forth in the following description section, which will become apparent through following description or will be understood through practice of the disclosure.

### BRIEF DESCRIPTION OF THE DRAWINGS

The above advantages and/or additional aspects and advantages of the disclosure will become apparent and easy to understand according to the attached figures, and

FIG. 1 is a flow chart of a food information processing method according to an embodiment of the disclosure;

FIG. 2 is a flow chart of a food information processing method according to another embodiment of the disclosure;

FIG. 3 is a structural diagram of a food information processing device according to an embodiment of the disclosure; and

FIG. 4 is a structural diagram of a food information processing device according to another embodiment of the disclosure.

### DETAILED DESCRIPTION

The embodiments of the disclosure are described in detail below, and examples of the embodiments are illustrated in the figures, and the same or similar mark numbers are used for throughout referring to the same or similar elements or elements with the same or similar functions. The embodiments described below with reference to the figures are illustrative and are intended to be used for explaining the disclosure rather than being construed as limitations of the disclosure.

A food information method and apparatus according to an embodiment of the disclosure are described below with reference to the figures.

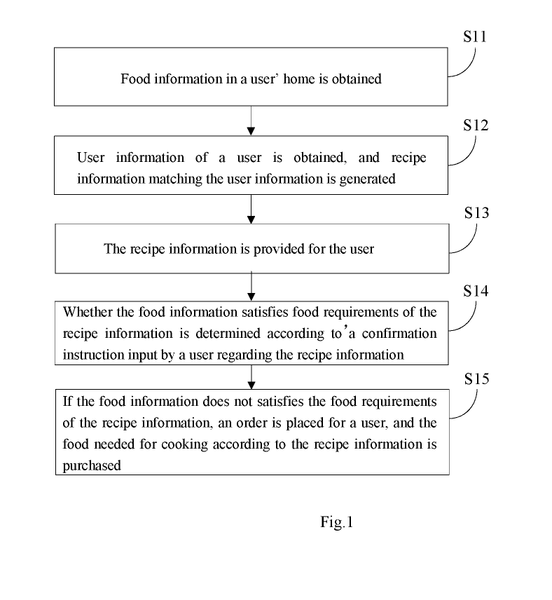

FIG. 1 is a flow chart of a food information processing method according to an embodiment of the disclosure.

As shown in FIG. 1, the food information processing method comprises the steps that:

S11. Food information in a user' home is obtained.

In one embodiment, the food information may comprise type and weight information of food.

In an embodiment of the disclosure, there are various ways to obtain the food information in the home of a user, as illustrated by the following examples:

As an example, the food information in the home of a user can be obtained through a camera in the home of the user.

As another example, the food information in the home of a user which is input through terminal equipment can be received, and the food information in the home of the user is determined based on the received information.

In one embodiment, the terminal equipment may be hardware equipment with various operating systems, such as a smart phone and a tablet computer, which is not limited by the embodiment.

It should be understood that when the food information in the home is input through a mobile terminal, the food information in the home is input by a user in a voice and/or text mode.

As another example, in the case of user authorization, a purchase record of food information purchased by a user online is obtained, and the food information in the home of a user is determined based on the purchase record.

As another example, food information in the home of a user is obtained from sensing information by communicating with a sensing device for scanning the food information in the home.

As another example, a picture which contains food information and is transmitted by a user through terminal equipment is received, and the food information in the home is obtained by analyzing the picture.

As another example, the food information in the home of a user may be obtained through household equipment such as a refrigerator.

For example, if a refrigerator in the home of a user is connected to the Internet of Things, the refrigerator can identify the food in the refrigerator, and related information of the food is uploaded to a server, so that the server obtains the food information in the refrigerator.

It should be understood that in practical applications, food information in the home of a user may be obtained through one or more of the above modes, the above modes are only examples of obtaining the food information in the home of the user, and the embodiment does not limit the modes of obtaining the food information in the home of the user.

S12. User information of a user is obtained, and recipe information matching the user information is generated.

In one embodiment, the user information comprises at least one of dietary habit information, health information and weight information.

As an example, the weight information of the user can be obtained through an intelligent weight scale in the home of the user.

As an example, the health information of the user can be obtained through equipment such as a blood pressure meter and a glucometer in the home of the user.

As an example, the health information and weight information of the user can be obtained according to a medical examination report of the user.

As an example, the dietary habit information of the user can be determined by a server based on historical cooking data of cooking equipment such as an electric pressure cooker in the home of the user.

It should be understood that the cooking data of the cooking equipment stored in the server can be uploaded to the server through the cooking equipment or can be uploaded by terminal equipment of the cooking equipment after the terminal equipment acquires the cooking data of the cooking equipment. In an embodiment of the disclosure, after the user information such as the dietary habit information (for example, taste preference), weight information and health information of the user is acquired, the recipe information matching the above information can be generated.

In one embodiment, the recipe information comprises recipes and cooking information.

In one embodiment, the cooking information may comprise, but not limited to, information such as the food amount, cooking time and cooking steps.

S13. The recipe information is provided for the user.

In an embodiment of the disclosure, the recipe information can be provided for a user in a variety of ways. For example, the recipe information can be provided for the user by means of text display and/or voice playing, image data and the like.

S14. Whether the food information satisfies food requirements of the recipe information is determined according to a confirmation instruction input by a user regarding the recipe information.

In an embodiment of the disclosure, after the recipe information is provided for a user, if the provided recipe information does not meet demands of the user, the user can send feedback information to a server through terminal equipment. Correspondingly, after receiving the feedback information sent by the terminal equipment, the server can adjust the recommended recipe information according to the feedback information, so that the adjusted recipe information meets the demands of the user.

It should be understood that when the recipe information provided for a user meets the demands, the user can send a confirmation instruction for the recipe information to a server through terminal equipment. Correspondingly, after receiving the confirmation instruction for the recipe information, the server can determine whether the food information in the home of the user satisfies the food requirements of the recipe information. Therefore, whether the food information in the home satisfies the food requirements of the recipe is judged, and the trouble for the user to determine whether the food in the home satisfies the food requirements of the recipe is reduced.

S15. If the food information does not satisfies the food requirements of the recipe information, an order is placed for a user, and the food needed for cooking according to the recipe information is purchased.

As an example, when it is determined that the food information in the home of a user does not satisfy the food requirements of the recipe, the e-commerce sales address corresponding to the needed food is obtained, and in the case of user authorization, payment is made based on a payment account bound by the user, so that the food needed for cooking according to the recipe information is purchased, and the time taken by the user to find the food and place the order is reduced.

In an embodiment of the disclosure, when it is determined that the food information in the home of a user does not meet the food material requirement of the recipe, link information for purchasing the food needed for cooking according to the recipe information can be provided for the user, so that whether the user purchases the food online is determined.

When a confirmation instruction from a user for the link information is received, an order is placed for the user, and the food needed for cooking according to the recipe information is purchased.

In addition, as an exemplary embodiment, when it is determined that the food information in the home of a user satisfies the food requirements of the recipe information, it can be prompted that the food information in the home of the user satisfies the food requirements of the recipe information.

In an embodiment of the disclosure, when it is determined that the food information in the home of a user satisfies the food requirements of the recipe information, in order to facilitate the user to view the recipe information in the cooking process, the recipe information can be pushed to cooking equipment, and the cooking equipment provides the recipe information for the user. For example, the cooking equipment can provides the recipe information for the user in a text display and/or voice playing mode.

According to the food information process method provided by the embodiment of the disclosure, the recipe information is generated according to the user information of a user, when the user confirms the recipe information, whether food information in the home of the user satisfies the food requirements of the recipe information is determined, when the food information in the home does not satisfies the food requirements of the recipe information, an order is placed for the user and the food needed for cooking according to the recipe information is purchased. Therefore, it is determined that whether the food information in the home of the user satisfies the food requirements of the recipe information, and the food needed for cooking according to the recipe information is purchased for the user, so that the trouble for the user to determine whether the food in the home satisfies the food requirements of the recipe is reduced, the time spent in purchasing the required food is shortened, and the user experience is effectively improved.

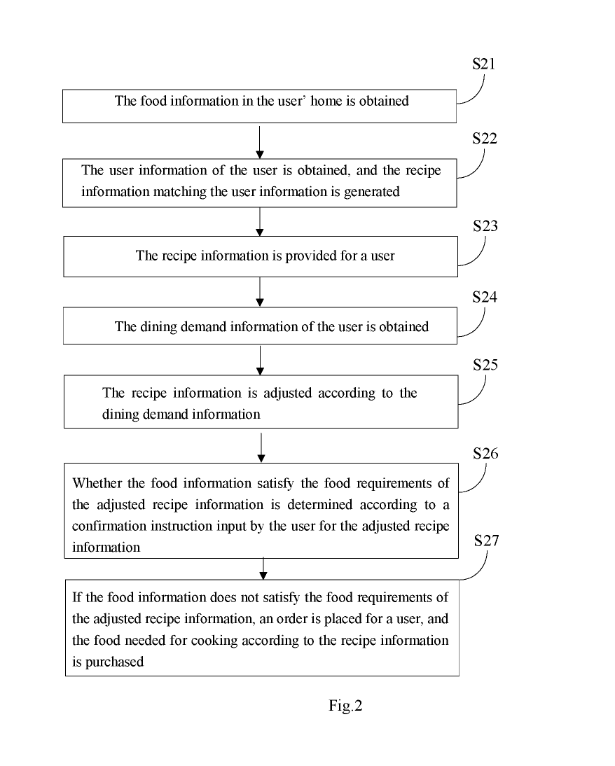

FIG. 2 is a flow chart of a food information processing method according to another embodiment of the disclosure. As shown in FIG. 2, the food information processing method comprises the steps that:

S21. The food information in the home of a user is obtained.

In one embodiment, the food information may comprise the type and weight of the food.

In an embodiment of the disclosure, the food information in the home of a user can be obtained in many modes, as illustrated by the following examples:

As an example, the food information in the home of a user can be obtained through a camera in the home of the user.

As another example, the food information in the home input by a user through terminal equipment can be received, and the food information in the home of the user is determined based on the received information.

In one embodiment, the terminal equipment may be hardware equipment with various operating systems, such as a smart phone and a tablet computer, which is not limited by the embodiment.

It should be understood that when the food information in the home is input through a mobile terminal, the food information in the home is input by a user in a voice and/or text mode.

As another example, in the case of user authorization, a purchase record of the food information purchased by a user online is obtained, and the food information in the home of a user is determined based on the purchase record.

As another example, the food information in the home of a user is obtained from sensing information by communicating with a sensing device for scanning the food information in the home.

As another example, a picture which contains the food information and is transmitted by a user through terminal equipment is received, and the food information in the home is obtained by analyzing the picture.

As another example, the food information in the home of a user may be obtained through household equipment such as a refrigerator.

For example, if a refrigerator in the home of a user is connected to the Internet of Things, the refrigerator can identify the food in the refrigerator, and related information of the food is uploaded to a server, so that the server obtains the food information in the refrigerator.

It should be understood that in practical applications, the food information in the home of a user may be obtained through one or more of the above modes, the above modes are only examples of obtaining the food information in the home of the user, and the embodiment does not limit the modes of obtaining the food information in the home of the user.

S22. The user information of the user is obtained, and the recipe information matching the user information is generated.

In one embodiment, the user information comprises at least one of dietary habit information, health information and weight information.

As an example, the weight information of the user can be obtained through an intelligent weight scale in the home of the user.

As an example, the health information of the user can be obtained through equipment such as a blood pressure meter and a glucometer in the home of the user.

As an example, the health information and weight information of the user can be obtained according to a medical examination report of the user.

As an example, the dietary habit information of the user can be determined by a server based on historical cooking data of cooking equipment such as an electric pressure cooker in the home of the user.

It should be understood that cooking data of the cooking equipment stored in the server can be uploaded to the server through the cooking equipment or can be uploaded by terminal equipment of the cooking device after the terminal equipment acquires the cooking data of the cooking equipment. In an embodiment of the disclosure, after the user information such as the dietary habit information (for example, taste preference), weight information and health information of the user is acquired, the recipe information matching the above information can be generated.

In one embodiment, the recipe information comprises recipes and cooking information.

In one embodiment, the cooking information may comprise, but not limited to, information such as the food amount, cooking time and cooking steps.

S23. The recipe information is provided for a user.

In an embodiment of the disclosure, the recipe information can be provided for a user in a variety of ways. For example, the recipe information can be provided for the user by means of text display and/or voice playing, image data and the like.

S24. The dining demand information of the user is obtained.

In one embodiment, the dining demand information comprises the number of diners and/or themes of dining demands.

In one embodiment, the number of diners and/or themes of dining demands uploaded by a user through terminal equipment are received.

In one embodiment, the themes of dining demands can comprise a vegetarian diet theme, a health keeping theme, an anti-hyperglycemia theme, an anti-hyperlipemia theme, an antihypertensive theme, a calcium supplementation theme and the like.

As an exemplary embodiment, in the using process of cooking equipment, a user can also upload dining demand information such as the number of diners and/or themes of dining demands to a server through the cooking equipment. Correspondingly, the server receives the dining demand information uploaded by the cooking equipment.

S25. The recipe information is adjusted according to the dining demand information.

In an embodiment of the disclosure, the recipes and/or cooking information can be adjusted based on the dining demand information.

For example, after the number of diners input by a user is received, the food amount in cooking information can be adjusted according to the number of diners, so that the food amount in the adjusted recipe information meets the requirement of the number of diners.

S26. Whether the food information satisfies the food requirements of the adjusted recipe information is determined according to a confirmation instruction input by a user for the adjusted recipe information.

In an embodiment of the disclosure, after the recipe information is provided for a user, if the provided recipe information does not meet demands of the user, the user can send feedback information to a server through terminal equipment. Correspondingly, after receiving the feedback information sent by the terminal equipment, the server can adjust the recommended recipe information according to the feedback information, so that the adjusted recipe information satisfies the demands of the user.

It should be understood that when the recipe information provided for a user meets the demands, the user can send a confirmation instruction for the recipe information to a server through terminal equipment. Correspondingly, after receiving the confirmation instruction for the recipe information, the server can judge whether the food information in the home of the user satisfies the food requirements of the recipe information. Therefore, whether the food information in the home satisfies the food requirements of the recipe is determined, and the trouble for the user to determine whether the food in the home satisfies the food requirements of the recipe is reduced.

S27. If the food information does not satisfy the food requirements of the adjusted recipe information, an order is placed for a user, and the food needed for cooking according to the recipe information is purchased.

As an example, when it is determined that the food information in the home of a user does not satisfy the food requirements of the recipe, the e-commerce sales address corresponding to the needed food is obtained, and in the case of user authorization, payment is made based on a payment account bound by the user, so that the food needed for cooking according to the recipe information is purchased, and the time taken by the user to find the food and place the order is reduced.

In an embodiment of the disclosure, when it is determined that the food information in the home of a user does not meet the food requirements of the recipe, link information for purchasing the food needed for cooking according to the recipe information can be provided for the user, so that whether the user purchase the food online is determined.

When a confirmation instruction from a user for the link information is received, an order is placed for the user, and the food needed for cooking according to the recipe information is purchased.

In addition, as an exemplary embodiment, when it is determined that the food information in the home of a user satisfies the food requirements of the recipe information, it can be prompted that the food information in the home of the user satisfies the food requirements of the recipe information.

In an embodiment of the disclosure, when it is determined that the food information in the home of a user satisfies the food requirements of the recipe information, in order to facilitate the user to view the recipe information in the cooking process, the recipe information can be pushed to cooking equipment, and the cooking equipment provides the recipe information for the user. For example, the cooking equipment can provides the recipe information for the user in a text display and/or voice playing mode.

In summary, according to the embodiment, the recipe information can be adjusted according to dining demand information, after a user confirms the adjusted recipe information, whether the food information in the home satisfies the food requirements of the adjusted recipe information is determined, an order is placed for the user, the food needed for cooking according to the recipe information is purchased. Thus, the food needed for cooking according to the recipe information is purchased for the user, the user can have enough food information for cooking according to the recipe, and the demands of the number of diners and/or themes of dining demands are met.

According to the food information process method provided by the embodiment of the disclosure, the recipe information is generated according to the user information of a user, when the user confirms the recipe information, whether the food information in the home of the user satisfies the food requirements of the recipe information is determined, when the food information in the home does not satisfies the food requirements of the recipe information, an order is placed for the user, and the food needed for cooking according to the recipe information is purchased. Therefore, it is determined that whether the food information in the home of the user satisfies the food requirements of the recipe information, and the food needed for cooking according to the recipe information is purchased for the user, so that the trouble for the user to determine whether the food in the home satisfies the food requirements of the recipe is reduced, the time spent in purchasing the required food is shortened, and the user experience is effectively improved.

In order to implement the embodiments, the disclosure further provides food information processing apparatus.

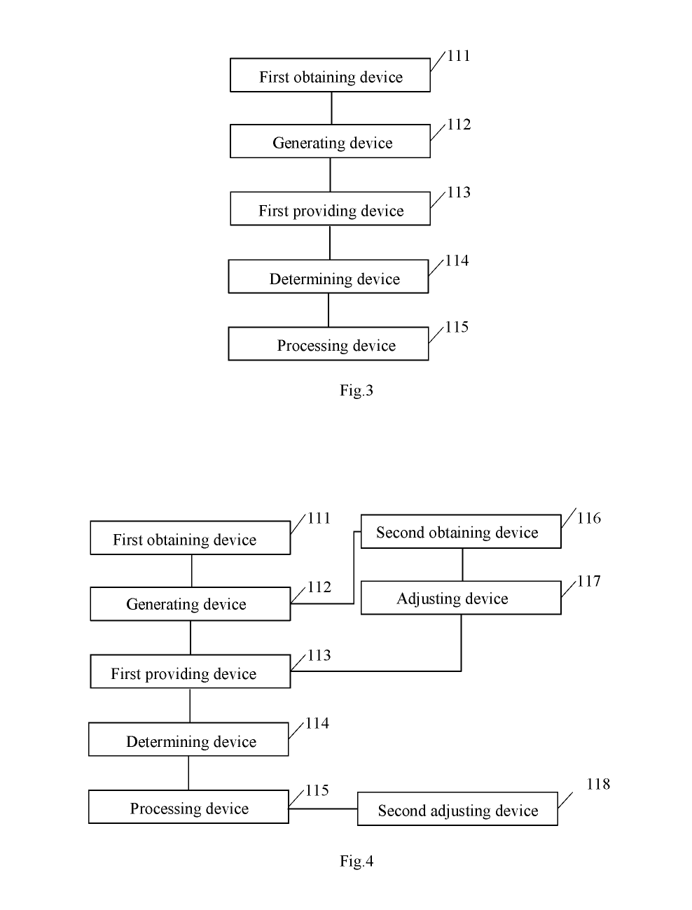

FIG. 3 is a structure diagram of food information processing apparatus according to an embodiment of the disclosure.

As shown in FIG. 3, the food information processing apparatus can further comprise a first obtaining device 111, a generating device 112, a first providing device 113, a determining device 114 and a processing device 115, and,

The first obtaining device 111 is used for obtaining food information in the home of a user.

In one embodiment, the food information may comprise type and weight of food.

The generating device 112 is used for obtaining user information of a user, and generating recipe information matching the user information.

In one embodiment, the user information comprises at least one of dietary habit information, health information and weight information.

The first providing device 113 is used for providing the recipe information for a user.

The determining device 114 is used for determining whether food information satisfies food requirements of recipe information according to a confirmation instruction input by a user for the recipe information.

The processing device 115 is used for ordering and purchasing the food needed for cooking according to the recipe information when the food information is determined not to meet the food requirements of the recipe information.

In an embodiment of the disclosure, in order to satisfy personalized requirements of a user, the device can further comprise, on the basis of FIG. 3, a second obtaining device 116 and an adjusting device 117, as shown in FIG. 4, and,

The second obtaining device 116 is used for obtaining dining demand information of a user;

The adjustment device 117 is used for adjusting the recipe information according to dining demand information so as to judge whether food information meets the food requirements of the adjusted recipe information.

In one embodiment, the dining demand information comprises the number of diners and/or themes of dining demands.

In an embodiment of the disclosure, a second obtaining device 116 is specifically used for receiving the number of diners and/or themes of dining demands uploaded by a user through terminal equipment.

In an embodiment of the disclosure, in order to prompt a user, as shown in FIG. 4, the device may further comprise a second providing device 118, and the second providing device 118 is used for providing the user with link information for purchasing the food needed for cooking according to the recipe information.

In one embodiment, the processing device 115 is used for ordering and purchasing the food needed for cooking according to the recipe information according to a confirmation instruction of the user for the link information.

It should be noted that the foregoing explanation of the embodiments of the food information process method is also applicable to the food information processing apparatus of the embodiment, the implementation principles of the method and the apparatus are similar, and details are not described herein again.

According to a food information processing device provided by the embodiment of the disclosure, recipe information is generated according to user information of a user, when the user confirms the recipe information, whether food information in the home of the user meets food requirements of the recipe information or not is determined, and an order is placed for the user and the food needed for cooking according to the recipe information is purchased when the food information in the home of the user does not satisfies the food requirements of the recipe information. Therefore, it is determined whether the food information in the home of the user satisfies the food requirements of the recipe information, and the food needed for cooking according to the recipe information is purchased, so that the trouble for the user to determine whether the food in the home satisfies the food requirements of a recipe is reduced, and the time spent in purchasing the required food is shortened, and the user experience is effectively improved.

According to description of the specification, description of the reference terms ‘an embodiment’, ‘some embodiments’, ‘examples’, ‘specific examples’ or ‘some examples’ and the like means that specific features, structures, materials or characteristics which are described in combination with the embodiments or examples are included in at least one embodiment or example of the disclosure. According to the specification, schematic representation of the above terms is not necessarily directed to the same embodiment or example. Furthermore, the described specific features, structures, materials or characteristics may be combined in a proper mode in any one or more embodiments or examples.

Moreover, the terms ‘first’ and ‘second’ are used for descriptive purposes only and are not to be construed as indicating or implying relative importance or implicitly indicating the number of indicated features. Thus, features defined with ‘first’ or ‘second’ may comprise at least one of the features explicitly or implicitly.

Any process or method description described in the flow charts or otherwise described herein may be understood to represent devices, segments or portion of codes which comprise one or more executable instructions for implementing particular logical functions or process steps, in addition, the scope of the embodiments of the disclosure includes additional implementations, in which the functions may be performed in a substantially simultaneous manner or in an opposite order depending on the functions involved without being performed in the shown or discussed order.

The logic and/or steps represented in the flow charts or otherwise described herein, for example, may be considered as an ordered list of executable instructions for implementing logical functions, and may be embodied in any computer readable medium for being used by an instruction execution system, a device or equipment (such as a computer-based system, a system comprising a processor or other systems which can obtain instructions from the instruction execution system, the device or the equipment and execute the instructions), or being used in combination with the instruction execution system, the device or the equipment. For the purposes of the specification, a ‘computer readable medium’ can be any device which may include, store, communicate, propagate or transmit programs for use by the instruction execution system, the device or the equipment or for use in combination with the instruction execution system, the device or the equipment. The computer readable medium specifically comprises following devices (a non-exhaustive list) such as an electrical connection part (an electronic device) with one or more wires, a portable computer disk box (a magnetic device), a random access memory (RAM), a read-only memory (ROM), an erasable editable read-only memory (EPROM or a flash memory), a fiber optic device and a portable compact disk read-only memory (CDROM). In addition, the computer readable medium may even be a piece of paper or other proper media on which the programs may be printed, because, for example, paper or other media can be optically scanned and then edited, interpreted or processed in other suitable mode when necessary for obtaining the programs electronically, and the programs are stored in a computer memory.

It should be understood that all parts of the disclosure may be implemented by hardware, software, firmware or a combination of hardware, software and firmware. According to the above-described embodiments, multiple steps or methods can be implemented by software or firmware stored in a memory and executed by a proper instruction execution system. For example, if implemented by hardware, as in another embodiment, the methods can be implemented by any one or combination of the following techniques which are well known in the art: a discrete logic circuit with logic gates for implementing logic functions on data signals, a specific integrated circuit with proper combinational logic gates, a programmable gate array (PGA), a field programmable gate array (FPGA) and the like.

In some embodiments all or part of the steps carried by the method of the above can be completed by a program instructing related hardware, the program can be stored in a computer readable storage medium, and the program comprises one or a combination of the steps of the method embodiments.

In addition, all functional units of each embodiment of the disclosure may be integrated into a processing device, or all the units can separately exist physically, or two or more units can be integrated into a device. The integrated device can be implemented in the form of hardware or in the form of a software functional device. An integrated device can also be stored in a computer readable storage medium if being implemented as a software functional device and sold or used as an independent product.

The mentioned storage medium may be a read-only memory, a magnetic disk or an optical disk or the like. Although the embodiments of the disclosure are shown and described, it should be understood that the described embodiments are illustrative and are not to be construed as limiting the scope of the disclosure. Changes, modifications, alterations and variations of the described embodiments may be made by those skilled in the art within the scope of the disclosure.

1. A food information processing method, comprising:
obtaining food information in a user's home from a user;
obtaining user information about the user, and generating recipe information matching the user information;
providing the recipe information for the user;
determining whether the food information satisfies food requirements of the recipe information according to a confirmation instruction input by the user regarding the recipe information; and
if not, then ordering and purchasing the food needed for cooking according to the recipe information for the user.

2. The method of claim 1, wherein the user information comprises at least one of dietary habit information, health information and weight information.

3. The method of claim 1, further comprising:
obtaining dining demand information of the user; and
adjusting the recipe information according to the dining demand information, and thus determining whether the food information satisfies the food requirements of the adjusted recipe information.

4. The method of claim 3, wherein the dining demand information comprises the number of diners and/or themes of dining demands, and the step of obtaining the dining demand information of the user comprises:
receiving the number of diners and/or the themes of dining demands which are uploaded by the user through terminal equipment.

5. The method of claim 1, before the step of ordering and purchasing the food needed for cooking according to the recipe information for the user, the method further comprises the steps of:
providing the user with link information for purchasing the food needed for cooking according to the recipe information; and
receiving a confirmation instruction of the user for the link information, and executing the step of ordering and purchasing the food needed for cooking according to the recipe information for the user.

6. Food information processing apparatus, comprising:
a first obtaining device used for obtaining food information in a user's home;
a generating device used for obtaining user information of the user and generating recipe information matching the user information;
a first providing device used for providing the recipe information for the user;
a determining device used for determining whether the food information satisfies food requirements of the recipe information according to a confirmation instruction input by the user regarding the recipe information; and
a processing device used for ordering and purchasing the food needed for cooking according to the recipe information for the user when the food information is determined not to satisfy the food requirements of the recipe information.

7. The apparatus of claim 6, wherein the user information comprises at least one of dietary habit information, health information and weight information.

8. The apparatus of claim 6, further comprising:
a second obtaining device used for obtaining dining demand information of the user; and
an adjustment device used for adjusting the recipe information according to the dining demand information so as to judge whether the food information satisfies the food requirements of the adjusted recipe information.

9. The apparatus of claim 8, wherein the dining demand information comprises the number of diners and/or themes of dining demands, and the second obtaining device is used for:
receiving the number of diners and/or the themes of dining demands which are uploaded by the user through terminal equipment.

10. The apparatus of claim 6, further comprising:
a second providing device used for providing the user with link information for purchasing the food needed for cooking according to the recipe information;
wherein the processing device is specifically used for ordering and purchasing the food needed for cooking according to the recipe information for the user according to a confirmation instruction of the user for the link information.
